(19) United States
(12) Patent Application Publication
(10) Pub. No.: US 2022/0197888 A1
(43) Pub. Date: Jun. 23, 2022

(54) DATA PROCESSING METHOD, APPARATUS, SYSTEM, DEVICE, AND STORAGE MEDIUM

(71) Applicant: BOE Technology Group Co., Ltd., Beijing (CN)

(72) Inventors: Hongliang WANG, Beijing (CN); Longfei LI, Beijing (CN); Xinxin LIU, Beijing (CN); Fuchen TIAN, Beijing (CN); Tongbo WANG, Beijing (CN)

(21) Appl. No.: 17/357,942

(22) Filed: Jun. 24, 2021

(30) Foreign Application Priority Data
Dec. 23, 2020 (CN) 202011538950.1

(51) Int. Cl. G06F 16/23; G06F 16/28

(52) U.S. Cl. CPC G06F 16/2358; G06F 16/2322; G06F 16/288; G06F 16/2365

(57) ABSTRACT

The present application discloses a data processing method, apparatus, system, device, and storage medium. The method includes: receiving IoT data reported by an IoT terminal device; according to a preset rule transmitted by a service system, determining target IoT data in the IoT data, wherein the preset rule is a condition for indicating storage processing of the IoT data; and storing the target IoT data in a storage location corresponding to the preset rule.

### CROSS-REFERENCE TO RELATED APPLICATION

The present application claims a priority of the Chinese patent application No. 202011538950.1 filed on Dec. 23, 2020, which is incorporated herein in its entirety.

### TECHNICAL FIELD

The present application relates to the field of Internet of Things technologies and specifically to the Internet of Things data processing technology, and in particular to a data processing method, apparatus, system, device and a storage medium.

### BACKGROUND

At present, basic data generated by enterprise application (APP) products, Internet of Things service systems, and health management systems, etc. are stored separately. If these basic data are stored in a unified data management platform, there is a privacy security problem for health and medical data.

### SUMMARY

According to a first aspect of the present application, a data processing method is provided and includes: receiving IoT data reported by an IoT terminal device; according to a preset rule transmitted by a service system, determining target IoT data in the IoT data, wherein the preset rule is a condition for indicating storage processing of the IoT data; and storing the target IoT data in a storage location corresponding to the preset rule.

In some embodiments, according to a preset rule transmitted by a service system, determining target IoT data in the IoT data, wherein the preset rule is a condition for indicating storage processing of the IoT data, includes: parsing the preset rule to obtain content corresponding to the preset rule; and determining the target IoT data in the IoT data according to the content corresponding to the preset rule.

In some embodiments, the content corresponding to the preset rule includes a target device identifier and a binding time parameter; the determining the target IoT data in the IoT data according to the content corresponding to the preset rule, includes: acquiring a reporting device identifier and a device uploading data time contained in the IoT data; in a case that the reporting device identifier is consistent with the target device identifier, screening the IoT data according to relationship between the device uploading data time and the binding time parameter to obtain the target IoT data.

In some embodiments, the content corresponding to the preset rule includes a target device identifier, a binding time parameter, a user attribute identifier and an analysis object condition; the determining the target IoT data in the IoT data according to the content corresponding to the preset rule, includes: parsing the preset rule to obtain the user attribute identifier, wherein the user attribute identifier is used for indicating whether to analyze the IoT data corresponding to the target device identifier; in a case that the user attribute identifier indicates performing data analysis on the IoT data corresponding to the target device identifier, analyzing the IoT data according to the analysis object condition, the target device identifier and the binding time parameter, to obtain an analysis processing result; determining the analysis processing result as the target IoT data.

In some embodiments, the analyzing the IoT data according to the analysis object condition, the target device identifier and the binding time parameter, includes: acquiring a reporting device identifier and a device uploading data time included in the IoT data; in a case that the reporting device identifier is consistent with the target device identifier and the device uploading data time is within the binding time parameter, screening the IoT data according to an analysis object range to obtain a screening result; performing analysis process on the screening result to obtain an analysis processing result.

In some embodiments, the method further includes: pushing the target IoT data to the service system according to the storage position corresponding to the preset rule.

In some embodiments, the method further includes: receiving IoT data reported by a service system; determining target IoT data in the IoT data according to a preset rule transmitted by the service system; storing the target IoT data to a storage location corresponding to the preset rule; pushing the target IoT data to the service system according to the storage location corresponding to the preset rule.

In some embodiments, there are at least two service systems, and the content corresponding to the preset rule includes a target device identifier, a binding time parameter and an analysis object condition; wherein according to a preset rule transmitted by a service system, determining target IoT data in the IoT data, includes: acquiring a reporting device identifier and a device uploading data time included in the IoT data; in a case that the reporting device identifier is consistent with a target device identifier of the at least two service systems, performing analysis process on the IoT data to obtain an analysis processing result; and determining the analysis processing result as the target IoT data.

According to a second aspect of the present application, a data processing method is provided and includes: obtaining a target storage condition, wherein the target storage condition includes a target device identifier and a binding time parameter; generating a preset rule according to the target storage condition, wherein the preset rule is a condition for indicating storage processing of IoT data; transmitting the preset rule to a data storage system.

In some embodiments, the target storage condition further includes a target user parameter and an analysis object range; wherein the generating the preset rule according to the target storage condition, includes: generating a user attribute identifier according to the target user parameter and the binding time parameter, wherein the user attribute identifier is configured to indicate whether to perform data analysis on IoT data corresponding to the target device identifier; generating the preset rule according to the target attribute identifier, the binding time parameter, the target device identifier and the analysis object range.

According to a third aspect of the present application, a computer device is provided and includes: a memory, a processor, and a computer program stored on the memory and executable on the processor, wherein the processor executes the program to implement: receiving IoT data reported by an IoT terminal device; according to a preset rule transmitted by a service system, determining target IoT data in the IoT data, wherein the preset rule is a condition for indicating storage processing of the IoT data; and storing the target IoT data in a storage location corresponding to the preset rule.

According to a fourth aspect of the present application, a computer device is provided and includes: a memory, a processor, and a computer program stored on the memory and executable on the processor, wherein the processor executes the program to implement the method of the second aspect.

It is to be understood that the contents in this section are not intended to identify the key or critical features of the embodiments of the present application, and are not intended to limit the scope of the present application. Other features of the present application will become readily apparent from the following description.

### BRIEF DESCRIPTION OF THE DRAWINGS

The drawings are included to provide a better understanding of the application and are not to be construed as limiting the application. Wherein:

FIG. 1a is an application scenario diagram according to an embodiment of the present application;

FIG. 1b is another application scenario diagram according to an embodiment of the present application;

FIG. 1c is still another application scenario diagram according to an embodiment of the present application;

FIG. 2 is a flowchart of a data processing method according to an embodiment of the present application;

FIG. 3 is a schematic diagram showing principle of a data processing method according to an embodiment of the present application;

FIG. 4 is a flowchart of another data processing method according to an embodiment of the present application;

FIG. 5 is a schematic diagram showing principle of another data processing method according to an embodiment of the present application;

FIG. 6 is a flowchart of another data processing method according to an embodiment of the present application;

FIG. 7 is a schematic diagram showing signaling interaction of another data processing method according to an embodiment of the present application;

FIG. 8 is a flowchart of still another data processing method according to an embodiment of the present application;

FIG. 9 is a flowchart of yet another data processing method according to an embodiment of the present application;

FIG. 10 is a flowchart of a specific example according to an embodiment of the present application;

FIG. 11 is a schematic diagram showing principle of another specific example according to an embodiment of the present application;

FIG. 12 is a flowchart of another data processing method according to an embodiment of the present application;

FIG. 13 is a schematic diagram showing data interaction of yet another data processing method according to an embodiment of the present application;

FIG. 14 is a schematic diagram showing data interaction of still yet another data processing method according to an embodiment of the present application;

FIG. 15 is a schematic diagram showing data interaction of still yet another data processing method according to an embodiment of the present application;

FIG. 16 is a schematic diagram showing principle of another data processing method according to an embodiment of the present application;

FIG. 17 is a schematic structural diagram of a data lake in a data processing method according to an embodiment of the present application;

FIG. 18 is a schematic structural diagram of a data processing apparatus according to an embodiment of the present application;

FIG. 19 is a schematic structural diagram of a data processing apparatus according to an embodiment of the present application;

FIG. 20 is a schematic structural diagram of a data processing system according to an embodiment of the present application; and

FIG. 21 is a schematic structural diagram of a computer system suitable for implementing a computer device or a server according to an embodiment of the present application.

### DETAILED DESCRIPTION

Reference will now be made in detail to the exemplary embodiments of the present application, examples of which are illustrated in the accompanying drawings, wherein the various details of the embodiments of the present application are included to facilitate understanding and are to be considered as exemplary only. Accordingly, a pedestrian skilled in the art should appreciate that various changes and modifications can be made to the embodiments described herein without departing from the scope and spirit of the present application. Also, descriptions of well-known functions and structures are omitted from the following description for clarity and conciseness.

It should be noted that in a case of no conflict, embodiments in the present application and features in the embodiments may be combined with each other. The present application will be described in detail hereinafter, with reference to the drawings and in conjunction with the embodiments.

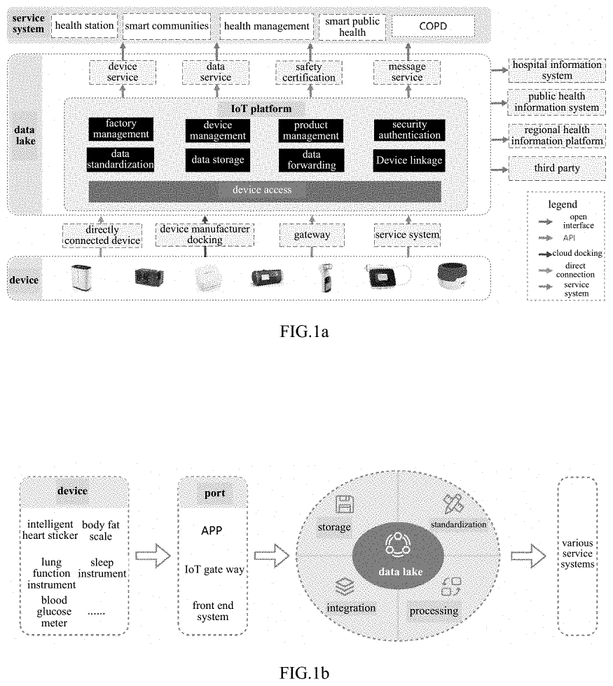

Referring to FIG. 1a to FIG. 1c, FIG. 1a to FIG. 1c are application scenario diagrams according to embodiments of the present application. As shown in FIG. 1a to FIG. 1c, FIG. 1a and FIG. 1b respectively show relationship between a data storage system, an access device and a service system from different perspectives of the data storage system, the access device and the service system. FIG. 1c describes data processing functions of the data storage system.

As shown in FIG. 1a, the device, the data storage system and the service system from are connected sequentially. The device may be a health detection device, such as a sphygmomanometer, a blood glucose meter, an intelligent heart sticker, a body fat scale, a lung function instrument, a sleep instrument, a body temperature detection device, a breast milk analyzer. The device is connected with the data storage system through an interface such as a direct connection device, cloud docking of device manufacturers, a gateway and the service system, so as to transmit collected user health data to the data storage system. The data storage system is an Internet of Things (IoT) data lake. The data storage system is configured with a plurality of function modules such as manufacturer management, device management, product management, security authentication, data standardization, data storage, data conversion, device linkage, so as to store, integrate, standardize and process data transmitted by the device. For example, it is determined through security authentication that a device for reporting data is a device that can receive Internet of Things data, then data uploaded by the device is processed by the data standardization module, and then is stored by the data storage module or is transmitted to the service system.

It should be understood that the data storage system builds a basic data ecosystem. By building a multi-source heterogeneous data one-stop development platform, the data storage system supports big data storage, calculation and analysis functions, such as data warehouse, interactive query, operation analysis, data visualization, search recommendation, real-time analysis, predictive analysis, thereby realizing the overall connection of data management and service in business operations. Specifically, as shown in FIG. 1c, the data lake is integrated with a micro-service framework, AI platform, big data computing functions, security & monitoring modules, multi-source heterogeneous data integration modules, hybrid cloud storage, data processing modules, etc. An outside of the data lake is connected with community supermarkets such as smart communities, health station, C-end & home such as mobile health, home IoT, B-end health management such as medical community, VIP health management, smart public health such as smart physical examination, smart follow-up, single-disease medical unit such as diabetes, hypertension, chronic obstructive pulmonary disease (COPD), online marketing. For data from different sources, regardless of these data transmitted to the data lake from any channel, as long as these data is corresponding to a same mobile phone number or a same ID number, the data lake backend should map these data to a user, and then push relevant required data according to the specific service system.

The service system is a platform system for data interaction with users, such as an enterprise APP, a health management system, a health station, a smart community, a health management, a smart public health. After the service systems receive data, they can independently process the data and push messages, etc.

It should be understood that data in the data storage system may also provide data support for a public welfare platform, such as a hospital information system, a public health information system, a regional health information platform.

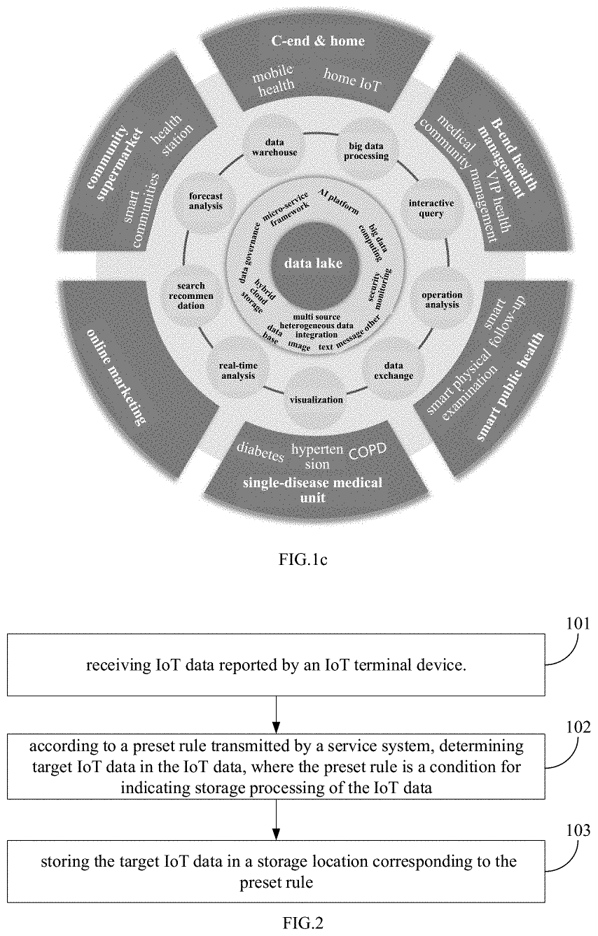

FIG. 2 is a flowchart of a data processing method according to an embodiment of the present application. It should be noted that an execution entity of the data processing method in this embodiment is a data processing apparatus which may be implemented by software and/or hardware. The data processing apparatus in this embodiment may be configured in a server which may be a data storage system, such as an IoT data lake.

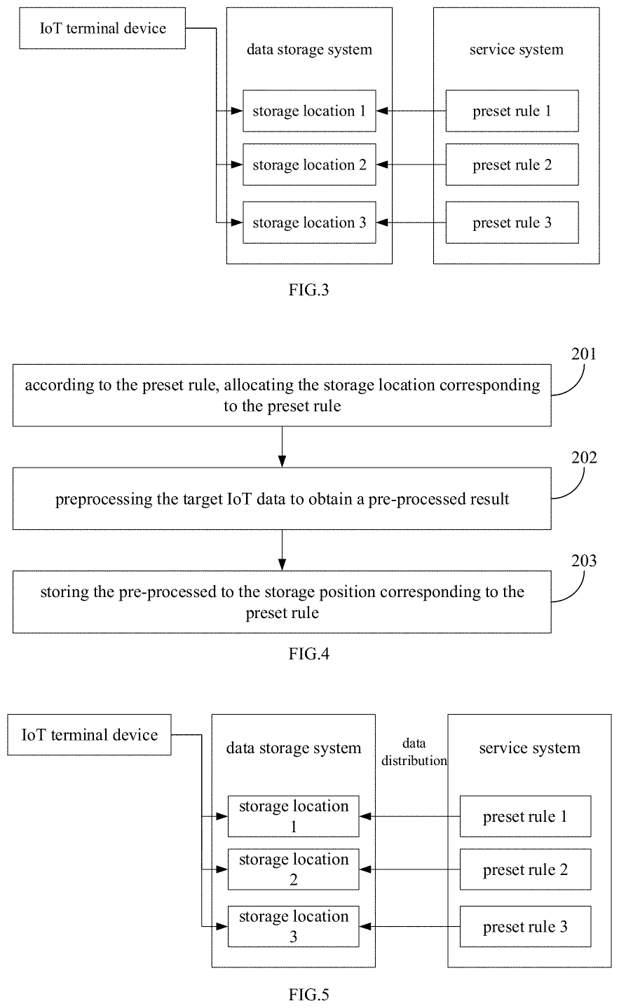

As shown in FIG. 2 and FIG. 3, the data processing method of this embodiment of the present application includes the following steps.

Step 101: receiving IoT data reported by an IoT terminal device.

It should be noted that the IoT terminal device includes a medical data collection device, such as a sphygmomanometer, a blood glucose meter. The IoT data at least includes user health data collected by the IoT terminal device. The IoT terminal device usually stores a network address of the data storage system. After the IoT terminal device detects the user health data, the IoT terminal device generates IoT data according to the user health data, and then reports the IoT data to the data storage system according to the network address of the data storage system.

It should be understood that the IoT terminal device according to embodiments of the present application is an IoT terminal device bound to the user when registering in a service system.

Step 102: according to a preset rule transmitted by a service system, determining target IoT data in the IoT data, where the preset rule is a condition for indicating storage processing of the IoT data.

It should be noted that the service system is a platform system for data interaction with users, such as an enterprise APP, a health management system. The service systems may be divided into a blood pressure system, a blood glucose system and the like, according to service types. The service systems may also be divided according to enterprises. Therefore, each service system, based on its own characteristics, generates a preset rule of condition for indicating storage processing of the IoT data, and then the service system transmits the preset rule to the data storage system, so that the data storage system determines target IoT data in the IoT data according to the preset rule transmitted by the service system. The target IoT data may be user health data collected by the IoT terminal device or an analysis result of the user health data.

It should be noted that, as shown in FIG. 17, the data storage system includes a plurality of function modules, such as a basic configuration module and a device access sub-module. The basic configuration module is configured to configure product types, management indexes, service system management information, configuration information and dictionary data. The device access sub-module is adapted with a plurality of fast access schemes, including common network environments and common transmission protocols, such as HTTP, Socket, MQTT, which can be adapted to data interaction between various IoT terminal devices and service systems, for example, receiving the IoT data transmitted by the IoT terminal device and the preset rule transmitted by the service system, etc. The address of the data storage system may be burned in the IoT terminal device.

It should be understood that the data storage system can only receive IoT data or preset rules from the IoT terminal device and the service system that have been in communication connection with the data storage system; and the IoT terminal device and the service system can receive data transmitted by the data storage system only after establishing connection with the data storage system. For the IoT terminal device that has been in communication connection with the data storage system, the data storage system can further provide corresponding device management service, such as management of device lifecycle for the IoT terminal device. One IoT terminal device may be associated with multiple service systems. For each service system, one IoT terminal device can only be associated with one institution, and one IoT terminal device may be associated with different users.

It should also be noted that some IoT terminal devices that do not have wireless communication with the data storage system, may also first use Bluetooth, local area network and other communication methods to transmit detected IoT data to a user terminal such as a smart phone loaded with a service system, and then the service system transmits the IoT data detected by the IoT terminal device to the data storage system.

As shown in FIG. 17, the data storage system may further include a data processing module. The data processing module may be further subdivided into a data receiving sub-module, a data conversion sub-module, a data distribution sub-module and a data storage sub-module. The data receiving sub-module is configured to receive data transmitted by a hardware device or data synchronized by the service system. The data conversion sub-module is configured to perform preprocessing and data standardization processing on received data. The data normalization processing refers to extracting useful information from device data and process it into data in a unified format. For example, extracted useful information is unified into structured data, stored in a relational database, and then may be queried and extracted through SQL statements.

The data distribution sub-module is configured to provide the service system with an ability to distribute data. For example, the following distribution modes can be supported between the data storage system and the service system:

a) communicating through a Message Queue (MQ);

b) direct Transmission over HTTP/HTTPS;

c) encrypted transmission over HTTP/HTTPS/RSA signature;

d) token mode.

As shown in FIG. 17, the data storage system may further include a data analysis module. The data analysis module may analyze data in a manner such as cloud computing, big data analysis, artificial intelligence algorithm models, and analysis results may be obtained in a manner including, but not limited to, real-time analysis, predictive analysis, etc.

In an embodiment of the present application, at least one IoT terminal device only needs to be configured with the network address of the data storage system, the at least one IoT terminal device can transmit IoT data to the data storage system, so that the data storage system can store IoT data reported by at least one IoT terminal device at the same time. Similarly, at least one service system only needs to be configured with the network address of the data storage system, the at least one service system can transmit a preset rule to the data storage system, so that the data storage system can store received IoT data according to the preset rule.

Step 103: storing the target IoT data in a storage location corresponding to the preset rule.

It should be noted that the data storage system has a distributed storage structure, that is, the target IoT data can be stored in a distributed manner according to the preset rule transmitted by the service system.

It should be understood that in a case that IoT data uploaded by one IoT terminal device meets preset rules transmitted by multiple service systems, the data storage system can store the IoT data according to each preset rule.

In this way, the data storage system of the present application can receive the preset rules of multiple service systems, and store the target IoT data to a storage position corresponding to each preset rule according to the preset rules of the multiple service systems, thereby effectively realizing multiplexing of the data storage system in the multiple service systems, effectively reducing cost of the data storage system and the service systems, and effectively avoiding data corresponding to the service systems becoming island data.

Optionally, since IoT data reported by each IoT terminal device usually has respective data characteristics, the IoT data needs to be pre-processed before storing the IoT data according to a preset rule

Specifically, as shown in FIG. 4, storing the target IoT data in the storage location corresponding to the preset rule, includes:

Step 201: according to the preset rule, allocating the storage location corresponding to the preset rule.

The storage location corresponding to the preset rule include, but not limited to, storage locations corresponding to service systems in a one-to-one manner, or, storage locations corresponding to types of user health data in a one-to-one manner, or, storage locations corresponding to health management needs in a one-to-one manner.

Step 202: preprocessing the target IoT data to obtain a pre-processed result.

It should be noted that due to factors such as manufacturers, types of data reported by various IoT terminal devices are usually different, including but not limited to, structured data, semi-structured data, un-structured data, etc. Therefore, in order to facilitate the data storage system to store and analyze the target IoT data, it is necessary to preprocess the target IoT data to unify formats of the target IoT data.

For example, regarding a user's blood pressure data, data formats of different types of IoT terminal devices produced by different manufacturers may be very different. For example, IoT data reported by an IoT terminal device 1 is a common character string “N12345H123L78”, where the numbers “12345” after “N” are a device number, the numbers “123” after “H” are a systolic pressure, the numbers “78” after “L” are a diastolic pressure; IoT data reported by an IoT terminal device 2 is a hexadecimal string “2711 7D 4A”, where “2711” are a device number, “7D” are a systolic pressure, and “4A” are a diastolic pressure. At this point, the IoT data reported by the IoT device 1 and the IoT data reported by the IoT terminal device 2 are unified in format. Optionally, the IoT data reported by the IoT terminal device 2 may be converted into a common character string with a conversion result as follows:

| systolic | diastolic | device | device binding | device unbinding |
|---|---|---|---|---|
| pressure | pressure | number | time | time |
| 123 | 78 | 12345 | 2020-01-03- | 2020-01-03- |
| | | | 10:22:37 | 10:26:45 |
| 125 | 74 | 10001 | 2020-04-12- | 2020-04-12- |
| | | | 18:28:52 | 18:31:03 |

Step 203: storing the pre-processed to the storage position corresponding to the preset rule.

In this way, the present application can perform data preprocessing on the IoT data reported by the IoT terminal device to facilitate the data storage system to store and analyze the target IoT data, thereby improving data processing speed of the data storage system.

Further, as shown in FIG. 5, the data processing method provided in the embodiment of the present application further includes: pushing the target IoT data to the service system according to the storage position corresponding to the preset rule.

In other words, the data storage system provided in the embodiment of the present application further has a function of distributing the stored target IoT data thereby meeting query and analysis needs of the service system on the target IoT data.

Specifically, the data storage system may distribute the target IoT data to the service systems in the following ways: a) communicating through a Message Queue (MQ), b) direct Transmission over HTTP/HTTPS, c) encrypted transmission over HTTP/HTTPS/RSA signature, d) token mode, e) API interface, etc.

It should be understood that the foregoing communication mode of distributing the target IoT data to the service systems by the data storage system may also be applied to a report process of the IoT terminal device to the data storage system.

Further, according to the preset rule transmitted by the service system, determining target IoT data in the IoT data, includes: parsing the preset rule to obtain content corresponding to the preset rule, and determining the target IoT data in the IoT data according to the content corresponding to the preset rule.

It should be noted that data in the health care field usually includes private data, for example, the health of users. Moreover, the service system and the data storage system are deployed separately, that is, the business service is a service platform deployed by an enterprise, and the data storage system is a data storage platform that can be shared by multiple enterprises. In order to keep privacy of its own users confidential, the service system usually does not share user information to the data storage platform, that is, user health data generated by measurement of the IoT terminal device is separated from the user information in the data storage system, thereby achieving the purpose of desensitizing the storage of user health data. In other words, the data storage system can only process and analyze the IoT data transmitted by the IoT terminal device, but cannot analyze user information. However, the data storage system not only has simple operation of storing and distributing the IoT data transmitted by the IoT terminal device, but also can perform data visualization processing, data fusion and data analysis on the stored user health data. Therefore, the present application proposes determining the target IoT data in the IoT data according to the preset rule transmitted by the service system.

As an optional embodiment, the content corresponding to the preset rule includes a target device identifier and a binding time parameter.

It should be noted that the target device identifier and the binding time parameter in the preset rule may be an identifier and time of an IoT terminal device used and/or bound when a user registers and/or logs in the service system. It should be understood that the binding time parameter may also include, but not limited to, time at which the IoT terminal device is unbound from the service system. Binding time and un-binding time are binding start time and binding end time between the user information and the target device identifier.

For example, when multiple users share one IoT terminal device such as a medical instrument in hospital, user information can be logged in an interaction interface of a service system pre-configured in the medical instrument before each use. At this point, the service system can acquire a target device identifier and a binding start time corresponding to the user information. When the current patient exits the user information at the end of use, an un-binding time is generated.

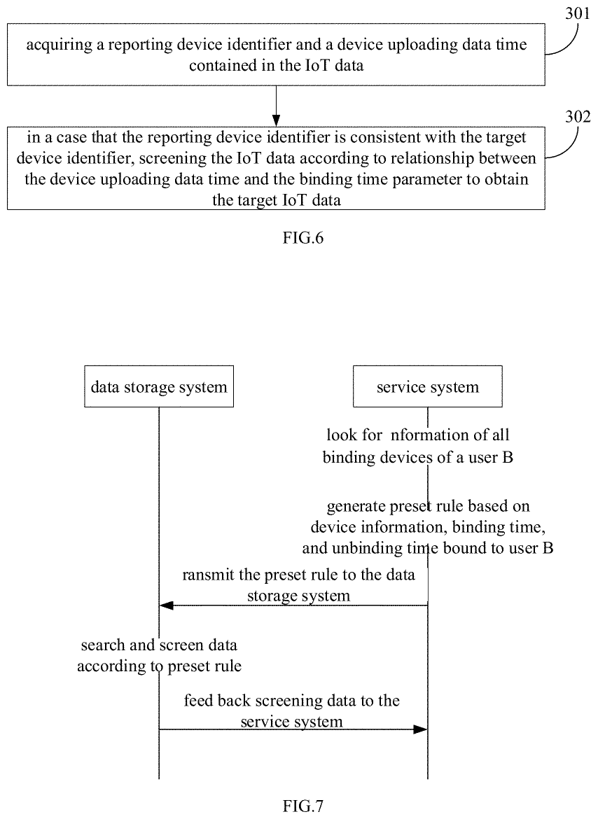

As shown in FIG. 6, according to the content corresponding to the preset rule, determining the target IoT data in the IoT data, includes:

Step 301: acquiring a reporting device identifier and a device uploading data time contained in the IoT data.

Since the IoT data is reported by the IoT terminal device, the reporting device identifier is a device identifier of an IoT terminal device that collects user health data. The device uploading data time is time at which the IoT terminal device reports the collected user health data to the data storage system. It should be understood that the device uploading data time refers to time at which the IoT terminal device initiates reporting, rather than time at which the data storage system receives the IoT data, thereby effectively avoiding errors caused by reporting delay and avoiding determination of valid data as invalid data.

Step 302: in a case that the reporting device identifier is consistent with the target device identifier, screening the IoT data according to relationship between the device uploading data time and the binding time parameter to obtain the target IoT data.

That is, when the reporting device identifier is consistent with the target device identifier, it means that the IoT terminal device that reports the IoT data to the data storage system is the same as the IoT terminal device used when the user registers and/or logs in the service system. At this point, the IoT data received by the data storage system is considered to be valid, and the IoT data can be screened according to the relationship between the device uploading data time and the binding time parameter, thereby obtaining the target IoT data.

Optionally, screening the IoT data according to relationship between the device uploading data time and the binding time parameter to obtain the target IoT data, includes, but not limited to, screening target IoT data that directly feeds back data and target IoT data that needs to be analyzed and processed and then fed back to data, from the IoT data.

For example, as shown in FIG. 7, the service system needs to look for user health data detected by all binding devices of a user B, such as a sphygmomanometer and a blood glucose meter. At this point, the service system searches for a device identifier having binding relationship with the user B and a corresponding binding time, from stored user information, to obtain the following information:

{
user ID: 123321,
SN: A123321,
binding_Time_Start: 2020-09-09-12:12:12,
binding_Time_End: 2020-09-09-14:12:12
}

Since one user may have multiple health detection devices, i.e., multiple pieces of foregoing information may be obtained from the query, each device number and corresponding binding time parameter may be further selected to generate a query list as follows:

[
{SN:..., time:2020-09-09(12:12:12-14:12:12)},
{SN:...,time:...},
...
]

The service system generates a preset rule according to the query list, and transmits the preset rule to the data storage system. According to the query list in the preset rule, the data storage system queries for data which simultaneously meets that any reporting device identifier is consistent with the target device identifier and the device uploading data time is within the binding time parameter, and takes the IoT data satisfying the query list as the target IoT data.

It should be understood that after the target IoT data is screened according to the query list, the queried IoT data may be directly transmitted to the service system, or the screened target IoT data may be further analyzed according to the preset rule transmitted by the service system.

As another optional embodiment, the content corresponding to the preset rule includes a target device identifier, a binding time parameter, a user attribute identifier, and an analysis object.

It should be noted that the target device identifier and the binding time parameter in the preset rule may be an identifier and time of an IoT terminal device used and/or bound when a user registers and/or logs in the service system. It should be understood that the binding time parameter may also include, but not limited to, time at which the IoT terminal device is unbound from the service system. Binding time and un-binding time are binding start time and binding end time between the user information and the target device identifier.

The user attribute identifier may be used to indicate whether to perform data analysis on the IoT data corresponding to the target device identifier, i.e., whether to perform data analysis on user health data of the user. For example, in a case that a user is an activate user of an additional health analysis function of the service system, the user may be labeled with a user attribute identifier to cause the data storage system to analyze the user's user health data.

An analysis object refers to data that needs to be analyzed. For example, in the field of blood pressure analysis, only blood pressure data that meets a hypertension criteria can be analyzed, such as analyzing the number and frequency of occurrence of hypertension.

For example, as shown in FIG. 8, determining the target IoT data in the IoT data according to the content corresponding to the preset rule, includes:

Step 401: parsing the preset rule to obtain a user attribute identifier, where the user attribute identifier is used for indicating whether to analyze the IoT data corresponding to the target device identifier.

As can be seen from the foregoing analysis, the user attribute identifier is determined by the service system according to the user information registered in the service system, and therefore, the user attribute identifier may be preset in the preset rule transmitted from the service system to the data storage system. Then, the user attribute identifier is obtained by parsing the preset rule.

For example, the user attribute identification may be a binary identifier. In a case that the user health data of the user needs to be analyzed, the user attribute identifier is as select=1. In a case that the user health data of the user does not need to be analyzed, he user attribute identifier is as select=0.

Step 402: in a case that the user attribute identifier indicates performing data analysis on the IoT data corresponding to the target device identifier, analyzing the IoT data according to an analysis object condition, the target device identifier and the binding time parameter, to obtain an analysis processing result.

Step 403: determining the analysis processing result as the target IoT data.

That is, in the embodiment of the present application, after receiving the preset rule transmitted by the service system, a user attribute identifier is first extracted from the preset rule. In a case that the user attribute identifier indicates performing data analysis on the IoT data corresponding to the target device identifier, an analysis object condition, a target device identifier and a binding time parameter are further acquired from the preset rule, and then analysis processing is performed on the IoT data reported by the IoT data terminal device to obtain an analysis processing result. In a case that the user attribute identifier indicates not performing data analysis on the IoT data corresponding to the target device identifier, only a storage location is extracted from the preset rule, so that the IoT data reported by the IoT terminal device is taken as the target IoT data and is stored according to the storage location in the preset rule, and then the target IoT data is pushed to the service system according to the storage location corresponding to the preset rule.

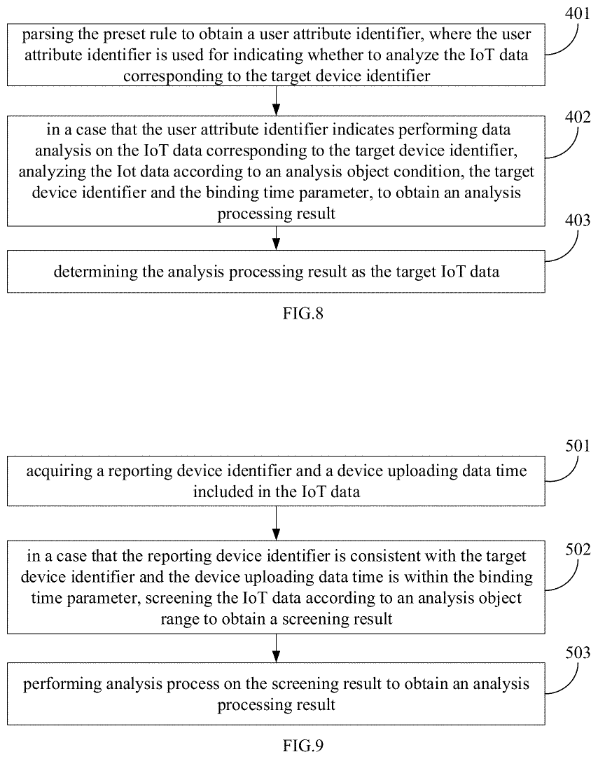

Further, as shown in FIG. 9, the step 402 of in a case that the user attribute identifier indicates performing data analysis on the IoT data corresponding to the target device identifier, analyzing the IoT data according to an analysis object condition, the target device identifier and the binding time parameter, to obtain an analysis processing result, further includes:

Step 501: acquiring a reporting device identifier and a device uploading data time included in the IoT data.

Since the IoT data is reported by the IoT terminal device, the reporting device identifier is a device identifier of an IoT terminal device that collects user health data. The device uploading data time is time at which the IoT terminal device reports the collected user health data to the data storage system. It should be understood that the device uploading data time refers to time at which the IoT terminal device initiates reporting, rather than time at which the data storage system receives the IoT data, thereby effectively avoiding errors caused by reporting delay and avoiding determination of valid data as invalid data.

Step 502: in a case that the reporting device identifier is consistent with the target device identifier and the device uploading data time is within the binding time parameter, screening the IoT data according to an analysis object range to obtain a screening result.

The device uploading data time being within the binding time parameter means that the device uploading data time is after the binding time when the user registers/logins the service system, and before the un-binding time when the user exits the service system. That is, in a case that the device uploading data time is between the binding time and the un-binding time, the device uploading data time is determined to be within the binding time parameter.

The analysis object range refers to a threshold for processing user health data. In other words, in a case that the user health data is in the analysis object range, the user health data is determined as a screening result.

Step 503: performing analysis process on the screening result to obtain an analysis processing result.

It should be understood that the analysis process performed on the screening result may also be preset according to the preset rule. That is, the preset rule may include an analysis rule instruction for analyzing the screening result. The data storage system may select a corresponding analysis model according to the analysis rule instruction. The analysis model may be a big data analysis model, AI analysis model, neural network analysis, etc., preset in the data storage system. Then the data storage system inputs the screening result into the corresponding analysis model to obtain the analysis processing result.

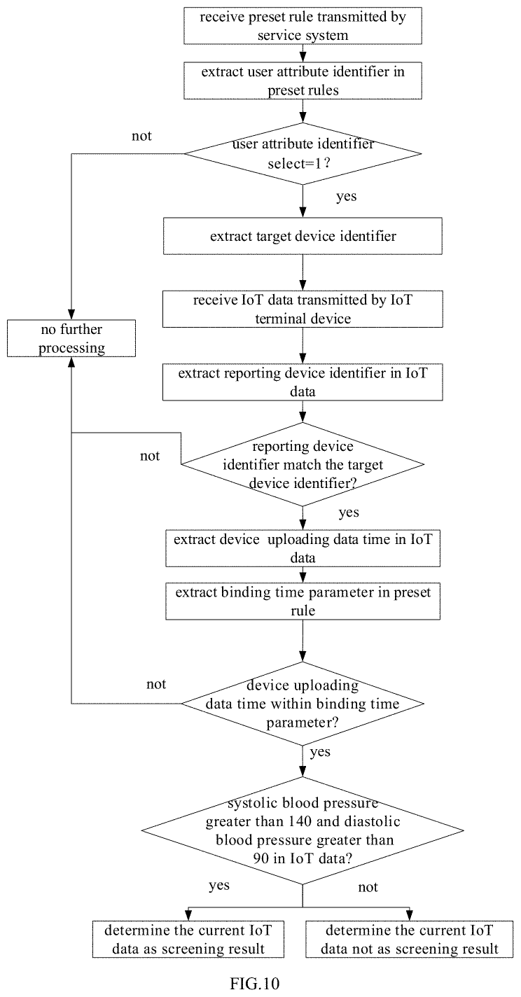

For example, as shown in FIG. 10, description is described herein after with an example that the service system needs to save a copy of newly uploaded IoT data which meets conditions of Chaoyang District, Beijing, female, age greater than 50, diastolic blood pressure greater than 90 and systolic blood pressure greater than 140, and return to the number of saved records and an average value of blood pressures.

When a user A is in a binding operation with a sphygmomanometer in a service system, a binding relationship is generated as follows:

{
user_ID:123456,
SN:A123456,
Binding_Time: 2020-09-08-12:12:12
}

Since user information is stored in the service system, the service system determines whether the user A is a user satisfying the conditions according to the user information stored in the service system. If the user A is a user satisfying the conditions, then, a user attribute identification of select=1 is generated for the user A. If the user A is not a user satisfying the conditions, then, a user attribute identification of select=0 is generated for the user A.

Then, according to the device number, the binding time and the user attribute identifier of the IoT terminal device bound by the user, the service system generates the following preset rule and transmits the following preset rule to the data storage system:

{
SN: A123456,
Binding_Time: 2020-09-08-12:12:12,
Select: 1,
blood pressure: diastolic blood pressure
greater than 90, systolic blood pressure greater
than 140
}

The data storage system analyzes the preset rule transmitted by the service system, and extracts a device identifier “SN: 123456” in the preset rule as the target device identifier in a case that the user attribute identifier is select=1, and further extracts a binding time parameter “Binding_Time: 2020-09-08-12:12:12”. The data storage system continues to receive IoT data transmitted by IoT devices, and extract a reporting device identifier in the data. In a case that the reporting device identifier is consistent with the target device identifier, i.e., the reporting device identifier is “SN: 123456”, then a binding time parameter in the preset rule and a device uploading data time in the IoT data are further acquired. In a case that the device uploading data time is within the binding time parameter, the device uploading data time is after 2020-09-08-12:12:12. It should be understood that an un-binding time is not recorded in the foregoing preset rule, thus, time after 2020-09-08-12:12:12 is within the binding time parameter. Then, user health data in the IoT data and an analysis object range in the preset rule are further acquired. In a case that a systolic blood pressure in the IoT data is greater than 140 and a diastolic blood pressure is greater than 90, then the current IoT data is determined as a screening result. In a case that a systolic blood pressure in the IoT data is less than or equal to 140 and a diastolic blood pressure is less than or equal to 90, then the current IoT data is not determined as a screening result.

In this way, the data processing method provided in the embodiment of the present application can use the data analysis model of the data storage system to analyze IoT data detected by the IoT terminal device, according to the preset rule transmitted by the service system, thereby providing the service system with intelligent auxiliary diagnosis results for corresponding data. For example, the data storage system can transmit results of real-time analysis and/or prediction to the service system, and the service system queries user information corresponding to the data in its own data, and then transmits an abnormal analysis result to a client interactive interface corresponding to the user information in time and/or transmits a reminder message to an account bound to the user information. In this way, the data processing method can realize detection and reminder of chronic diseases such as hypertension, diabetes and chronic respiratory diseases. As an optional embodiment, the service system may also directly transmit to-be-analyzed data to the data storage system, so that an analysis model preset in the data storage system can be used to perform data analysis on the to-be-analyzed data, thereby effectively solving the problem of insufficient data analysis capabilities of the service system. For example, some institution (not limited to physical examination institutions) may bind and measure devices provided by the institution, aggregate measurement data onto a terminal device of the institution, and then the terminal device transmits data to the data storage system over a wired network or a wireless network.

Specifically, the data storage system receives the IoT data reported by the service system; determines target IoT data in the IoT data according to the preset rule transmitted by the service system; stores the target IoT data to a storage position corresponding to the preset rule; and pushes the target IoT data to the service system according to the storage position corresponding to the preset rule.

For example, for a fundus picture captured by a fundus camera of a physical examination institution, the picture and a preset rule including analysis requirement are transmitted to the data storage system through a service system preset in the fundus camera. Then, the data storage system extracts the to-be-analyzed fundus picture and the analysis requirement, respectively, and inputs the fundus picture into an analysis model corresponding to the analysis requirement, thereby realizing analysis of the fundus picture, and obtaining an analysis result. The data storage system transmits the analysis result to the service system.

As another example, in a case that a user uses a breast milk analyzer, a preset rule transmitted by a service system to a data storage system may be to recommend recipes based on breast milk. After receiving breast milk detection data transmitted by an IoT terminal device, the data storage system can transmit the breast milk detection data to a recipe recommendation model, so that the recipe recommendation model analyzes the breast milk detection data to obtain targeted recipes recommended for mothers. Then, the data storage system feeds back the analyzed recipes to the service system, so that the service system can recommend recipes to mothers.

For another example, the data storage system may receive image data of a medical report transmitted by the service system, then perform optical character recognition (OCR) on the image data of the medical report to extract character data in the medical report, and feed back it to the service system.

As yet another optional embodiment, there are at least two service systems, and the content corresponding to the preset rule includes a target device identifier, a binding time parameter and an analysis object condition, then, according to the content corresponding to the preset rule, determining the target IoT data in the IoT data, includes: acquiring a reporting device identifier and a device uploading data time included in the IoT data; in a case that the reporting device identifier is consistent with a target device identifier of the at least two service systems, performing analysis process on the IoT data to obtain an analysis processing result; and determining the analysis processing result as the target IoT data.

As shown in FIG. 11, the data storage system may further solve the problem of data fusion among multiple service systems. Specifically, in a case that a service system 1 and a service system 2 want to acquire each other's data or use the other's existing data for data analysis, when data sharing is allowed at decision-making levels of the two service systems, the service system 1 and the service system 2 can respectively transmit to the data storage system a permission of transferring and/or copying data stored in accordance with their original preset rules to a new shared location. For example, data A, B, C of the service system 1 may be copied to the shared location, and data D, E of the service system 2 may be copied to the shared location; then, according to a new preset rule, the data storage system performs fusion analysis to the data A, B, C, D, and E in the shared location, and transmits an analysis results to the service system 1 and the service system 2, respectively. It should be understood that the service system 1 and the service system 2 may also individually instruct the data storage system to perform a personalized analysis on the IoT data in the shared location according to their own service requirements.

In data fusion analysis, data generated by different channels, different service systems, and different types of hardware devices may be transmitted to the data storage system for fusion analysis through multiple transmission methods. The foregoing transmission methods include, but not limited to, transmitting data to the data storage system through an IoT hardware device directly connected to the network, or directly according to a burned address of a server that receives data.

Alternatively, hardware devices that use wireless communication methods such as Bluetooth may be bound to the device through an APP or applet installed in a terminal device such as a mobile phone, and the transmitted data is transmitted to the APP background and then is transmitted to the data storage system through an APP background data interface. After data processing, data can be distributed to various service systems, or after data analysis, AI big data processing, etc., a report or result is returned to the user. In some institutions (not limited to physical examination institutions), a user can use a device provided in the institution to bind and measure data. The data is collected on the institution's terminal device, and then the terminal device transmits these data to the data storage system through wired and wireless communication.

For example, the service system 1 may be a platform for screening of diabetic retinopathy (which is a complication of diabetes), although current AI algorithm can reach more than 90% accuracy, there are still cases where it is judged as a false positive. If it can be known that a screened person is not suffering from diabetes, then a result of screening for glycoreticulum can be corrected. Data provided by the service system 2 can include diabetes prevalence of the user who have been screened for glycoreticulum with the service system. In view of this, the data storage system can well screen the device identifier of the user with diabetes according to the service system 1 and the service system 2.

When multiple service systems jointly use data in the data storage system, the data in the shared location may be stored with a latest data table and/or all data table. The latest data table is stored according to the device identifier, service system, index, and stores the last measured data. The all data table stores all data in the data storage system. In the data storage system, the latest data corresponding to the multiple service systems can be viewed (queried). The data storage system can feed back latest data detected by a corresponding device to one service system according to a preset rule of the one service system. It should be understood that what a user sees in a terminal device (not limited to an applet, APs, H5 page) is all the latest data measured by himself, regardless of source, device, or institution. In conclusion, the data storage system of the present application can receive preset rules of the multiple service systems and store the target IoT data to a storage position corresponding to the preset rules according to the preset rules of the multiple service systems, thereby effectively multiplexing of the data storage system in the multiple service systems, effectively reducing cost of the data storage system and the service systems, and effectively avoiding data corresponding to the service systems becoming island data.

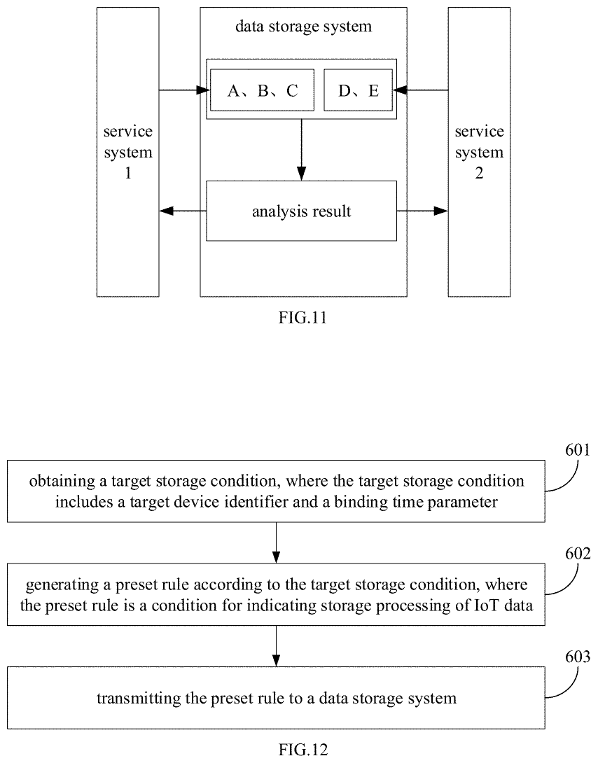

FIG. 12 is a flowchart of another data processing method according to an embodiment of the present application. It should be noted that an execution body of the data processing method of this embodiment is a service system. The service system can perform data interaction with a data storage system. As shown in FIG. 12, the data processing method according to this embodiment of the present application includes the following steps.

Step 601: obtaining a target storage condition, where the target storage condition includes a target device identifier and a binding time parameter;

Step 602: generating a preset rule according to the target storage condition, where the preset rule is a condition for indicating storage processing of IoT data;

Step 603: transmitting the preset rule to a data storage system.

As an optionally embodiment, the method further includes:

the target storage condition further includes a target user parameter and an analysis object range, then, generating the preset rule according to the target storage condition, includes:

generating a user attribute identifier according to the target user parameter and the binding time parameter, where the user attribute identifier is configured to indicate whether to perform data analysis on IoT data corresponding to the target device identifier;

generating the preset rule according to the target attribute identifier, the binding time parameter, the target device identifier and the analysis object range.

It should be noted that details not disclosed in the data processing method of this embodiment of the present application may refer to details disclosed in the foregoing embodiments of the present application.

In conclusion, the data storage system of the present application can receive the preset rules of multiple service systems, and store the target IoT data to a storage position corresponding to each preset rule according to the preset rules of the multiple service systems, thereby effectively realizing multiplexing of the data storage system in the multiple service systems, effectively reducing cost of the data storage system and the service systems, and effectively avoiding data corresponding to the service systems becoming island data.

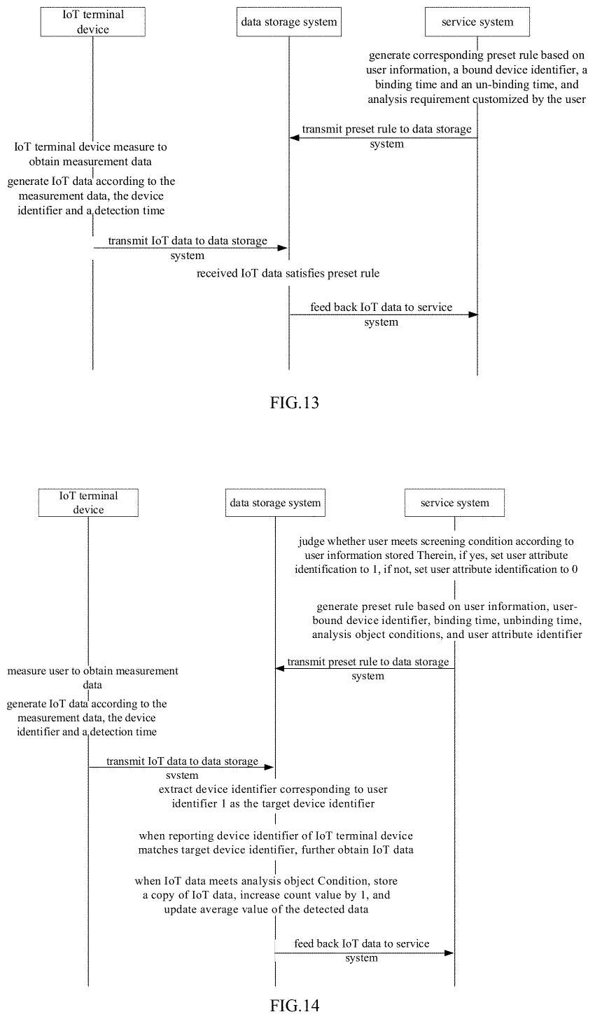

Data processing methods are described hereinafter in connection with FIG. 13 to FIG. 17. FIG. 13 is a flowchart of another data processing method according to an embodiment of the present application. The data processing method provided in this embodiment of the present application is applied to a data processing system. The data storage system takes an IoT data lake as an example. FIG. 16 is a schematic diagram showing interaction between the data lake and the service system provided in the embodiment of the present application.

As shown in FIG. 13, in a service system, a user is bound to an IoT terminal device (i.e., a detection device). The service system generates a corresponding preset rule based on user information, a bound device identifier, a binding time and an un-binding time, and analysis requirement customized by the user. The service system transmits the preset rule to the data storage system. The IoT terminal device (i.e., the detection device) measure the user to obtain measurement data. Then, the IoT terminal device generates IoT data according to the measurement data, the device identifier and a detection time, and transmits the IoT data to the data storage system. In a case that the received IoT data satisfies the preset rule, the data storage system stores the IoT data to a storage position corresponding to the preset rule. In a case that the received IoT data does not satisfy the preset rule, the data storage system performs no processing on the IoT data.

Further, as shown in FIG. 14, the service system determines whether the user meets a screening condition according to user information stored therein. If the user meets the screening condition, the service system sets a user attribute identifier as 1, and if not, the service system sets a user attribute identifier as 0. The service system generates a preset rule according to the user information, the device identifier bound by the user, the binding time, the unbinding time, the analysis object condition and the user attribute identifier, and transmits the preset rule to the data storage system. The IoT terminal device (i.e., the detection device) generates IoT data according to the measurement data, the device identifier and a detection time, and transmits the IoT data to the data storage system. The data storage system extracts a device identifier corresponding to a user identifier 1 as a target device identifier. In a case that the reporting device identifier of the IoT terminal device is consistent with the target device identifier, the data storage system further acquires the IoT data. In a case that the IoT data conforms to the analysis object condition, the data storage system stores a copy of the IoT data, a count value is incremented by one. The data storage system updates a detection data average value, and returns a result to the service system.

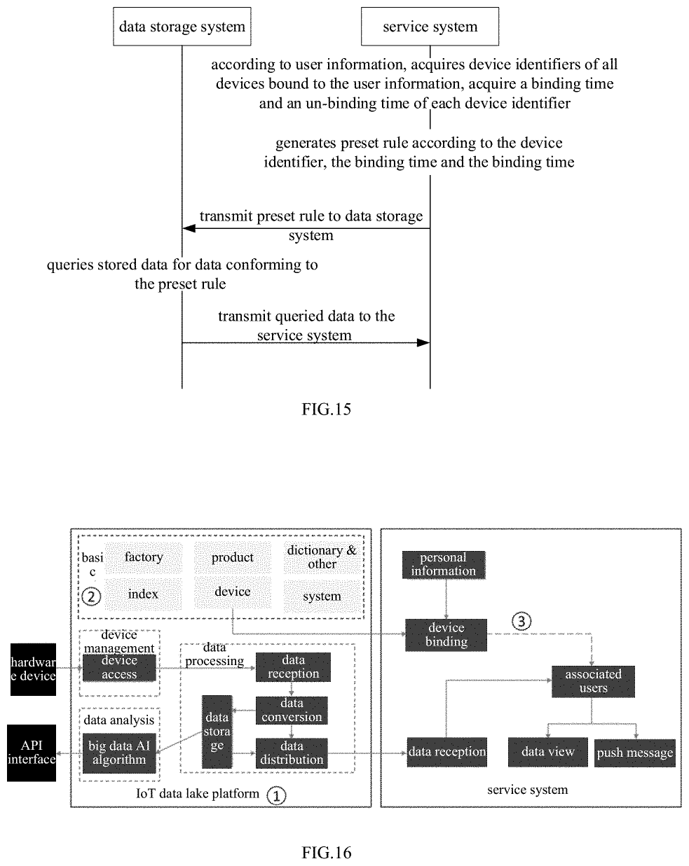

Alternatively, as shown in FIG. 15, according to user information, the service system acquires device identifiers of all devices bound to the user information, acquires a binding time and an un-binding time of each device identifier, generates a preset rule according to the device identifier, the binding time and the binding time, and transmits the preset rule to the data storage system. The data storage system queries the stored data for data conforming to the preset rule and transmits the queried data to the service system.

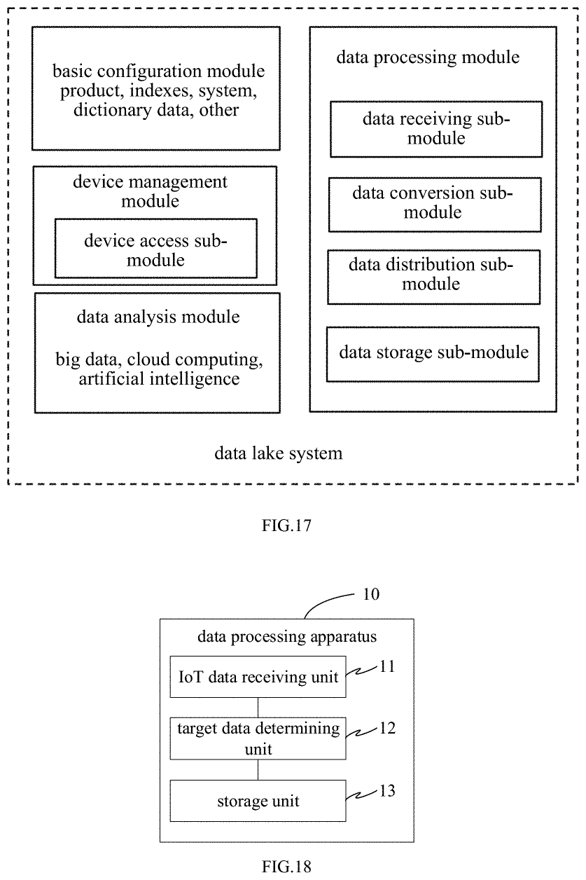

FIG. 18 is a schematic structural diagram of a data processing apparatus according to an embodiment of the present application. The data processing apparatus may be configured in a server which is a data storage system, such as an IoT data lake. As shown in FIG. 18, the data processing apparatus 10 includes:

an IoT data receiving unit 11 configured to receive IoT data reported by an IoT terminal device;

a target data determining unit 12 configured to, according to a preset rule transmitted by a service system, determine target IoT data in the IoT data, where the preset rule is a condition for indicating storage processing of the IoT data;

a storage unit 13 configured to store the target IoT data in a storage location corresponding to the preset rule.

In some embodiments, the target data determining unit 12 is further configured to:

parse the preset rule to obtain content corresponding to the preset rule;

determine the target IoT data in the IoT data according to the content corresponding to the preset rule.

In some embodiments, the target data determining unit 12 is further configured to:

acquire a reporting device identifier and a device uploading data time contained in the IoT data;

in a case that the reporting device identifier is consistent with the target device identifier, screen the IoT data according to relationship between the device uploading data time and the binding time parameter to obtain the target IoT data.

In some embodiments, the target data determining unit 12 is further configured to:

parse the preset rule to obtain a user attribute identifier, where the user attribute identifier is used for indicating whether to analyze the IoT data corresponding to the target device identifier;

in a case that the user attribute identifier indicates performing data analysis on the IoT data corresponding to the target device identifier, analyze the IoT data according to an analysis object condition, the target device identifier and the binding time parameter, to obtain an analysis processing result.

determine the analysis processing result as the target IoT data.

In some embodiments, the target data determining unit 12 is further configured to:

acquire a reporting device identifier and a device uploading data time included in the IoT data;

in a case that the reporting device identifier is consistent with the target device identifier and the device uploading data time is within the binding time parameter, screen the IoT data according to an analysis object range to obtain a screening result;

perform analysis process on the screening result to obtain an analysis processing result.

In some embodiments, the storage unit 13 is further configured to:

according to the preset rule, allocate the storage location corresponding to the preset rule;

preprocess the target IoT data to obtain a pre-processed result;

store the pre-processed to the storage position corresponding to the preset rule.

In some embodiments, the storage unit 13 is further configured to:

push the target IoT data to the service system according to the storage position corresponding to the preset rule.

In some embodiments, the data processing apparatus 10 is further configured to:

receive IoT data reported by a service system;

determine target IoT data in the IoT data according to a preset rule transmitted by the service system;

store the target IoT data to a storage location corresponding to the preset rule;

push the target IoT data to the service system according to the storage location corresponding to the preset rule.

In some embodiments, there are at least two service systems, and the content corresponding to the preset rule includes a target device identifier, a binding time parameter and an analysis object condition. Then, when determining the target IoT data in the IoT data according to the content corresponding to the preset rule, the data processing apparatus 10 is further configured to:

acquire a reporting device identifier and a device uploading data time included in the IoT data;

in a case that the reporting device identifier is consistent with a target device identifier of the at least two service systems, perform analysis process on the IoT data to obtain an analysis processing result; and

determine the analysis processing result as the target IoT data.

It should be noted that details not disclosed in the data processing apparatus of this embodiment of the present application may refer to details disclosed in the foregoing embodiments of the present application.

It should be understood that the units or modules recited in the data storage apparatus 10 are corresponding to various steps in the method described with reference to FIG. 2. Thus, the operations and features described above with respect to the method are equally applicable to the data storage apparatus and the units contained therein, which are not repeated herein.

For the several modules or units mentioned in the foregoing detailed description, such division is not mandatory. In fact, according to the embodiments of the present disclosure, features and functions of two or more modules or units described above may be embodied in one module or unit. Conversely, features and functions of a module or unit described above may be further divided into multiple modules or units.

In conclusion, the data storage system of the present application can receive the preset rules of multiple service systems, and store the target IoT data to a storage position corresponding to each preset rule according to the preset rules of the multiple service systems, thereby effectively realizing multiplexing of the data storage system in the multiple service systems, effectively reducing cost of the data storage system and the service systems, and effectively avoiding data corresponding to the service systems becoming island data.

FIG. 19 is a schematic structural diagram of a data processing apparatus according to an embodiment of the present application. The data processing apparatus is disposed in a service server. The service server performs data interaction with a data storage system through a data processing apparatus. The data processing apparatus may be a service system. As shown in FIG. 19, the data processing apparatus 20 includes:

a storage condition obtaining unit 21 configured to acquire a target storage condition, where the target storage condition includes a target device identifier and a binding time parameter;

a rule generation unit 22 configured to generate a preset rule according to the target storage condition, where the preset rule is a condition for indicating storage processing of IoT data;

a rule transmission unit 23 configured to transmit the preset rule to a data storage system.

In some embodiments, the storage condition acquisition unit 21 is further configured to:

generate a user attribute identifier according to the target user parameter and the binding time parameter, where the user attribute identifier is configured to indicate whether to perform data analysis on IoT data corresponding to the target device identifier;

generating the preset rule according to the target attribute identifier, the binding time parameter, the target device identifier and the analysis object range.

It should be understood that the units or modules recited in the data processing apparatus are corresponding to various steps in the method described with reference to FIG. 12. Thus, the operations and features described above with respect to the method are equally applicable to the data processing apparatus and the units contained therein, which are not repeated herein.

For the several modules or units mentioned in the foregoing detailed description, such division is not mandatory. In fact, according to the embodiments of the present disclosure, features and functions of two or more modules or units described above may be embodied in one module or unit. Conversely, features and functions of a module or unit described above may be further divided into multiple modules or units.

The functional units in the various embodiments of the present application may be integrated into one processing unit, or each unit may exist alone physically, or two or more units may be integrated into one unit. The foregoing integrated unit may be implemented in the form of hardware or software functional unit.

In conclusion, the data storage system of the present application can receive the preset rules of multiple service systems, and store the target IoT data to a storage position corresponding to each preset rule according to the preset rules of the multiple service systems, thereby effectively realizing multiplexing of the data storage system in the multiple service systems, effectively reducing cost of the data storage system and the service systems, and effectively avoiding data corresponding to the service systems becoming island data.

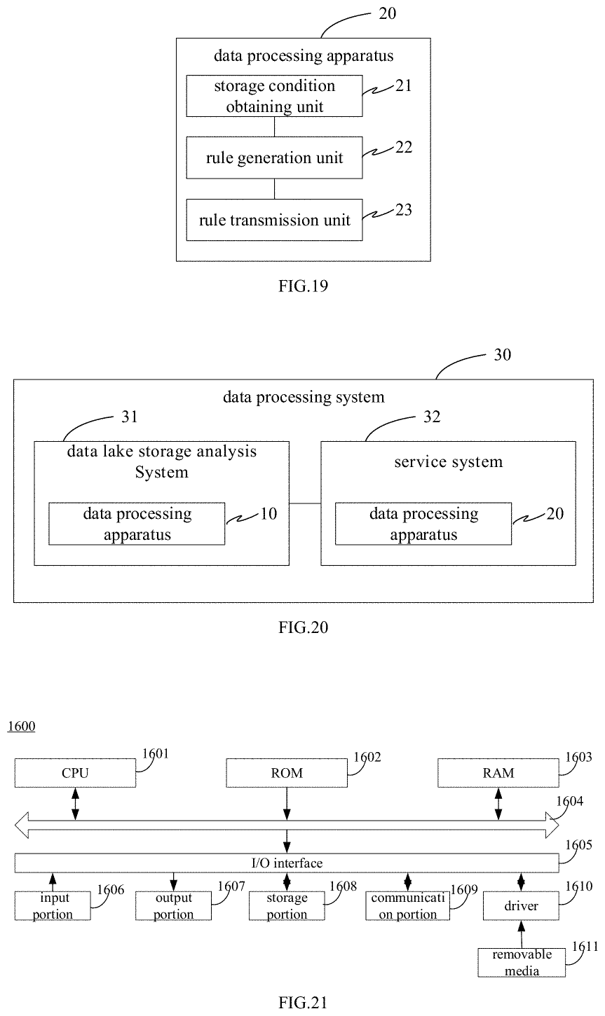

FIG. 20 is a schematic structural diagram of a data processing system according to an embodiment of the present application. As shown in FIG. 20, the data processing system 30 includes: a data lake storage analysis system 31 and the at least one service systems 32.

The data lake storage analysis system 31 includes a data storage apparatus 10. The data lake storage analysis system 31 is configured to store IoT data uploaded by an IoT terminal device;

The service system 32 includes a data storage apparatus 20. The service system 32 is configured to store user data in a binding relationship with an IoT terminal device.

It should be understood that the units or modules recited in the data storage apparatus 10 and the data storage apparatus 20 are corresponding to various steps in the method described with reference to FIG. 2. Thus, the operations and features described above with respect to the method are equally applicable to the data storage apparatus 10 and the data storage apparatus 20 and the units contained therein, which are not repeated herein.

For the several modules or units mentioned in the foregoing detailed description, such division is not mandatory. In fact, according to the embodiments of the present disclosure, features and functions of two or more modules or units described above may be embodied in one module or unit. Conversely, features and functions of a module or unit described above may be further divided into multiple modules or units.

Referring to FIG. 21, it shows a schematic structural diagram of a computer system 1600 suitable for implementing a computer device or a server according to an embodiment of the present application.

As shown in FIG. 21, the computer system 1600 includes a central processing unit (CPU) 1601 that can perform various suitable actions and processes in accordance with a program stored in a read-only memory (ROM) 1602 or a program loaded into a random access memory (RAM) 1603 from a storage portion 1608. In the RAM 1603, various programs and data required for operation of the system 1600 are also stored. The CPU 1601, the ROM 1602, and the RAM 1603 are connected to each other through a bus 1604. An input/output (I/O) interface 1605 is also connected to the bus 1604.

The input/output (I/O) interface 1605 is connected with the following components including: an input portion 1606 including a keyboard, a mouse, etc.,; an output portion 1607 including a cathode ray tube (CRT), a liquid crystal display (LCD), and a speaker, etc.; a storage portion 1608 including a hard disk, etc.,; and a communication portion 1609 including a network interface card, such as a LAN card, a modem. The communication portion 1609 performs communication processing via a network, such as the Internet. A driver 1610 may also be connected to the I/O interface 1605 as desired. Removable media 1611, such as magnetic disks, optical disks, magneto-optical disks, semiconductor memories, etc. may be mounted on the driver 1610 as desired so that computer programs read therefrom are installed into the storage portion 1608 as desired.

In particular, according to embodiments of the present disclosure, the process described above with reference to FIG. X may be implemented as a computer software program. For example, one embodiment of the present disclosure provides a computer program product including a computer program tangibly embodied on a machine-readable medium. The computer program includes program codes for performing the method of FIG. 2 or FIG. 12. In such embodiments, the computer program may be downloaded and installed from the network through the communication portion 1609 and/or installed from the removable medium 1611.

The flowchart and block diagrams in the drawings illustrate the architecture, functionality, and operation of possible implementations of systems, methods, and computer program products according to various embodiments of the invention. In this regard, each block in the flowchart or block diagrams may represent a module, a program segment, or a portion of codes, which includes one or more executable instructions for implementing the specified logical function(s). It should also be noted that, in some alternative implementations, the functions noted in the block may occur out of the order noted in the figures. For example, two blocks represented in succession may, in fact, be executed substantially concurrently, or the blocks may sometimes be executed in the reverse order, depending upon the functionality involved. It will also be noted that each block of the block diagrams and/or flowchart, and combinations of blocks in the block diagrams and/or flowchart, may be implemented by special purpose hardware-based systems that perform the specified functions or acts, or combinations of special purpose hardware and computer instructions.

In another aspect, the present application further provides a computer-readable storage medium. The computer-readable storage medium may be a computer-readable storage medium included in the apparatus described in the foregoing embodiments. The computer-readable storage medium may also be separately present, not assembled into the device. The computer-readable storage medium stores one or more programs that are executed by one or more processors to perform data processing methods described herein

The above are merely the embodiments of the present disclosure and shall not be used to limit the scope of the present disclosure. It should be noted that, a pedestrian skilled in the art may make improvements and modifications without departing from the principle of the present disclosure, and these improvements and modifications shall also fall within the scope of the present disclosure. The protection scope of the present disclosure shall be subject to the protection scope of the claims.

What is claimed is:

1. A data processing method, comprising:
receiving IoT data reported by an IoT terminal device;
according to a preset rule transmitted by a service system, determining target IoT data in the IoT data, wherein the preset rule is a condition for indicating storage processing of the IoT data; and
storing the target IoT data in a storage location corresponding to the preset rule.

2. The method according to claim 1, wherein according to a preset rule transmitted by a service system, determining target IoT data in the IoT data, wherein the preset rule is a condition for indicating storage processing of the IoT data, includes:
parsing the preset rule to obtain content corresponding to the preset rule; and
determining the target IoT data in the IoT data according to the content corresponding to the preset rule.

3. The method according to claim 2, wherein the content corresponding to the preset rule includes a target device identifier and a binding time parameter; the determining the target IoT data in the IoT data according to the content corresponding to the preset rule, includes:
acquiring a reporting device identifier and a device uploading data time contained in the IoT data;
in a case that the reporting device identifier is consistent with the target device identifier, screening the IoT data according to relationship between the device uploading data time and the binding time parameter to obtain the target IoT data.

4. The method according to claim 2, wherein the content corresponding to the preset rule includes a target device identifier, a binding time parameter, a user attribute identifier and an analysis object condition; the determining the target IoT data in the IoT data according to the content corresponding to the preset rule, includes:
parsing the preset rule to obtain the user attribute identifier, wherein the user attribute identifier is used for indicating whether to analyze the IoT data corresponding to the target device identifier;
in a case that the user attribute identifier indicates performing data analysis on the IoT data corresponding to the target device identifier, analyzing the IoT data according to the analysis object condition, the target device identifier and the binding time parameter, to obtain an analysis processing result;
determining the analysis processing result as the target IoT data.

5. The method according to claim 4, wherein the analyzing the IoT data according to the analysis object condition, the target device identifier and the binding time parameter, includes:
acquiring a reporting device identifier and a device uploading data time included in the IoT data;
in a case that the reporting device identifier is consistent with the target device identifier and the device uploading data time is within the binding time parameter, screening the IoT data according to an analysis object range to obtain a screening result;
performing analysis process on the screening result to obtain an analysis processing result.

6. The method according to claim 1, wherein the storing the target IoT data in a storage location corresponding to the preset rule, includes:
according to the preset rule, allocating the storage location corresponding to the preset rule;
preprocessing the target IoT data to obtain a pre-processed result;
storing the pre-processed to the storage position corresponding to the preset rule.

7. The method according to claim 1, further comprising:
pushing the target IoT data to the service system according to the storage position corresponding to the preset rule.

8. The method according to claim 1, further comprising:
receiving IoT data reported by a service system;
determining target IoT data in the IoT data according to a preset rule transmitted by the service system;
storing the target IoT data to a storage location corresponding to the preset rule;
pushing the target IoT data to the service system according to the storage location corresponding to the preset rule.

9. The method according to claim 1, wherein there are at least two service systems, and the content corresponding to the preset rule includes a target device identifier, a binding time parameter and an analysis object condition;
wherein according to a preset rule transmitted by a service system, determining target IoT data in the IoT data, includes:
acquiring a reporting device identifier and a device uploading data time included in the IoT data;
in a case that the reporting device identifier is consistent with a target device identifier of the at least two service systems, performing analysis process on the IoT data to obtain an analysis processing result; and
determining the analysis processing result as the target IoT data.

10. A data processing method, comprising:
obtaining a target storage condition, wherein the target storage condition includes a target device identifier and a binding time parameter;
generating a preset rule according to the target storage condition, wherein the preset rule is a condition for indicating storage processing of IoT data;
transmitting the preset rule to a data storage system.

11. The method according to claim 10, wherein the target storage condition further includes a target user parameter and an analysis object range;
wherein the generating the preset rule according to the target storage condition, includes:
generating a user attribute identifier according to the target user parameter and the binding time parameter, wherein the user attribute identifier is configured to indicate whether to perform data analysis on IoT data corresponding to the target device identifier;
generating the preset rule according to the target attribute identifier, the binding time parameter, the target device identifier and the analysis object range.

12. A computer device, comprising: a memory, a processor, and a computer program stored on the memory and executable on the processor, wherein the processor executes the program to implement:
receiving IoT data reported by an IoT terminal device;
according to a preset rule transmitted by a service system, determining target IoT data in the IoT data, wherein the preset rule is a condition for indicating storage processing of the IoT data; and
storing the target IoT data in a storage location corresponding to the preset rule.

13. The computer device according to claim 12, wherein the processor executes the program to implement:
parsing the preset rule to obtain content corresponding to the preset rule; and
determining the target IoT data in the IoT data according to the content corresponding to the preset rule.

14. The computer device according to claim 13, wherein the processor executes the program to implement:
acquiring a reporting device identifier and a device uploading data time contained in the IoT data;
in a case that the reporting device identifier is consistent with the target device identifier, screening the IoT data according to relationship between the device uploading data time and the binding time parameter to obtain the target IoT data.

15. The computer device according to claim 13, wherein the content corresponding to the preset rule includes a target device identifier, a binding time parameter, a user attribute identifier and an analysis object condition; the processor executes the program to implement:
parsing the preset rule to obtain the user attribute identifier, wherein the user attribute identifier is used for indicating whether to analyze the IoT data corresponding to the target device identifier;
in a case that the user attribute identifier indicates performing data analysis on the IoT data corresponding to the target device identifier, analyzing the IoT data according to the analysis object condition, the target device identifier and the binding time parameter, to obtain an analysis processing result;
determining the analysis processing result as the target IoT data.

16. The computer device according to claim 15, wherein the processor executes the program to implement:
acquiring a reporting device identifier and a device uploading data time included in the IoT data;
in a case that the reporting device identifier is consistent with the target device identifier and the device uploading data time is within the binding time parameter, screening the IoT data according to an analysis object range to obtain a screening result;
performing analysis process on the screening result to obtain an analysis processing result.

17. The computer device according to claim 12, wherein the processor executes the program to implement:
according to the preset rule, allocating the storage location corresponding to the preset rule;
preprocessing the target IoT data to obtain a pre-processed result;
storing the pre-processed to the storage position corresponding to the preset rule.

18. The computer device according to claim 12, wherein the processor executes the program to implement:
receiving IoT data reported by a service system;
determining target IoT data in the IoT data according to a preset rule transmitted by the service system;
storing the target IoT data to a storage location corresponding to the preset rule;
pushing the target IoT data to the service system according to the storage location corresponding to the preset rule.

19. The computer device according to claim 12, wherein there are at least two service systems, and the content corresponding to the preset rule includes a target device identifier, a binding time parameter and an analysis object condition; the processor executes the program to implement:
acquiring a reporting device identifier and a device uploading data time included in the IoT data;
in a case that the reporting device identifier is consistent with a target device identifier of the at least two service systems, performing analysis process on the IoT data to obtain an analysis processing result; and
determining the analysis processing result as the target IoT data.

20. A computer device, comprising: a memory, a processor, and a computer program stored on the memory and executable on the processor, wherein the processor executes the program to implement the method according to claim 10.
